(19) United States
(12) Patent Application Publication
(10) Pub. No.: US 2023/0371866 A1
(43) Pub. Date: Nov. 23, 2023

(54) Blood Draw Device with In-Line Diversion Volume for Collection of a Blood Culture Sample from the PIV During Indwell

(71) Applicant: Becton, Dickinson and Company, Franklin Lakes, NJ (US)

(72) Inventor: Jonathan Karl Burkholz, Salt Lake City, UT (US)

(21) Appl. No.: 18/198,557

(22) Filed: May 17, 2023

Related U.S. Application Data
(60) Provisional application No. 63/343,752, filed on May 19, 2022.

(51) Int. Cl. A61B 5/15

(52) U.S. Cl. CPC A61B 5/150992; A61B 5/15003; A61B 5/150213

(57) ABSTRACT

A blood draw system including a blood draw device having an actuator and a blood draw tube operably coupled to the actuator, the actuator configured to selectively advance the blood draw tube through a catheter of a vascular access device when the blood draw device is coupled to the vascular access device. The system also includes an extension tube extending from the blood draw device, the proximal extension tube being in fluid communication with the blood draw tube, and a proximal connector positioned at a proximal end of the extension tube. Furthermore, the system includes an initial blood volume diversion device positioned in-line with the extension tube between the blood draw device and the proximal connector, wherein the initial blood volume diversion device includes a diversion and isolation chamber configured to receive and hold an initial volume of blood drawn through the extension tube by the blood draw device.

### CROSS-REFERENCE TO RELATED APPLICATION

The present application claims priority to U.S. Provisional Application No. 63/343,752 entitled “Blood Draw Device with In-Line Diversion Volume for Collection of a Blood Culture Sample From the PIV During Indwell” filed May 19, 2022, the entire disclosure of which is hereby incorporated by reference in its entirety.

### BACKGROUND OF THE INVENTION

### Field of the Invention

The present disclosure generally relates to systems and methods for collecting a blood sample for blood culture testing from vascular access devices such as, e.g., peripheral intravenous catheters (PIVCs). More particularly, the systems described herein include blood draw devices with an in-line vented blood diversion chamber.

### Description of Related Art

When collecting a blood specimen from an indwelling vascular access devices, like a peripheral IV catheter (PIVC), a central venous catheter (CVC), or a peripherally inserted central catheter (PICC), the first 1-10 ml of blood is often wasted (i.e., disposed of) to avoid contamination from fluids in the dead space of the vascular access path of such devices. This extra step, while reducing contamination of the blood specimen, may be forgotten or improperly performed, thus compromising the blood specimen.

Blood cultures are often used as a tool to detect the presence of bacteria or fungi in a blood sample of a patient, to identify the type of bacteria or fungi present, and to direct the treatment of the patient. However, accidental contamination of the blood sample is a common problem, causing false positives and often resulting in a patient being prescribed unnecessary treatments such as, e.g., broad spectrum antibiotics. To address this concern, some healthcare providers clean the skin of the patient prior to a blood draw procedure. While this reduces the false positive rate, the rate is still significant (e.g., 3-5%) due to bacteria and/or fungi residing in, e.g., hair follicles. Therefore, some systems also divert a small volume of the initial blood drawn, with the initial (and potentially contaminated) volume being discarded. These systems, however, can be costly and time-consuming, and may only be used with an intravenous catheter at the immediate time of initial placement. Furthermore, these systems often rely on puncturing a patient's skin to collect the sample, which is uncomfortable for the patient.

Additionally, needle-free blood draw systems, such as PIVO™ from Velano Vascular, Inc., are intended to be used in conjunction with indwelling intravenous catheters within the patient's vasculature to draw one or more blood samples directly from the vascular access device, avoiding the need for additional (and uncomfortable) venipunctures. However, insertion of the intravenous catheter into the patient's vasculature may introduce bacteria and/or fungi by contact with the patient's skin and dermal layers during the insertion process. Accordingly, the initial blood volume drawn into the needle-free blood draw system may contain the bacteria and/or fungi present solely due to catheter insertion, thereby increasing the risk of a false positive blood culture test.

### SUMMARY OF THE INVENTION

In accordance with an aspect of the present disclosure, a blood draw system is disclosed, the blood draw system including a blood draw device having a distal end portion and a proximal end portion, wherein the blood draw device includes an actuator and a blood draw tube operably coupled to the actuator, wherein the actuator is configured to selectively advance the blood draw tube through a catheter of a vascular access device when the blood draw device is coupled to the vascular access device. The blood draw system also includes an extension tube extending from the proximal end portion of the blood draw device, wherein the proximal extension tube is in fluid communication with the blood draw tube of the blood draw device, and a proximal connector positioned at a proximal end of the extension tube. The blood draw system further includes an initial blood volume diversion device positioned in-line with the extension tube between the blood draw device and the proximal connector, wherein the initial blood volume diversion device includes a diversion and isolation chamber configured to receive and hold an initial volume of blood drawn through the extension tube by the blood draw device.

In some embodiments, the initial blood volume dispersion device further includes a venting portion in fluid communication with the diversion and isolation chamber.

In some embodiments, the venting portion is configured to vent air when the blood draw device is coupled to the vascular access device and the blood draw tube of the blood draw device is advanced into a patient's vasculature to draw the initial volume of blood into the diversion and isolation chamber.

In some embodiments, the venting portion is configured to vent air automatically when the blood draw device is coupled to the vascular access device and the blood draw tube of the blood draw device is advanced into a patient's vasculature.

In some embodiments, the venting portion is manually vented when the blood draw device is coupled to the vascular access device and the blood draw tube of the blood draw device is advanced into a patient's vasculature.

In some embodiments, the venting portion is formed of at least one of a membrane, paper, porous material, film, or mechanical feature that allows air to pass therethrough but prevents fluid to pass therethrough when wetted.

In some embodiments, the initial blood volume diversion device further includes a flashback visualization and blood sample fluid path arm, and the flashback visualization and blood sample fluid path arm is in fluid communication with the extension tube and the proximal connector.

In some embodiments, the initial blood volume diversion device further includes a distal fluid path diversion adapter and a proximal vented collar adapter, and the diversion and isolation chamber extends between the distal fluid path diversion adapter and the proximal vented collar adapter.

In some embodiments, the proximal vented collar adapter includes a vented portion, and a proximal end of the diversion and isolation chamber terminates at the vented portion.

In some embodiments, the initial blood volume diversion device further includes a primary flow tube extending between the distal fluid path diversion adapter and the proximal vented collar adapter and positioned parallel to the diversion and isolation chamber, wherein the primary flow tube is in fluid communication with the extension tube and the proximal connector.

In some embodiments, the initial blood volume diversion device further includes a fluid occlusion device positioned at a distal portion of the diversion and isolation chamber to selectively occlude the flow of blood from the diversion and isolation chamber.

In some embodiments, the proximal connector is configured to be removably coupled to a luer lock access device.

In some embodiments, the proximal connector is integrated with a luer lock access device.

In some embodiments, the blood draw device further includes an introducer body, and the actuator is configured to move linearly along the introducer body to advance and retract the blood draw tube from the distal end portion of the blood draw device.

In some embodiments, the diversion and isolation chamber has an internal volume of at least 0.15 mL.

Accordingly to another aspect of the present disclosure, a method of using a blood sample collection system is disclosed. The method includes providing the blood sample collection system, the system including a blood draw device having a distal end portion and a proximal end portion, wherein the blood draw device includes an actuator and a blood draw tube operably coupled to the actuator, wherein the actuator is configured to selectively advance the blood draw tube, an extension tube extending from the proximal end portion of the blood draw device, wherein the proximal extension tube is in fluid communication with the blood draw tube of the blood draw device, a blood collection interface positioned at a proximal end of the extension tube, and an initial blood volume diversion device positioned in-line with the extension tube between the blood draw device and the blood collection interface, wherein the initial blood volume diversion device includes a diversion and isolation chamber configured to receive and hold an initial volume of blood drawn through the extension tube by the blood draw device. The method also includes coupling the blood draw device to a vascular access device having an indwelling catheter, advancing the blood draw tube of the blood draw device through the vascular access device and beyond a distal tip of the indwelling catheter, and drawing the initial volume of blood through the blood draw tube and the extension tube and into the diversion and isolation chamber of the initial blood volume diversion device.

In some embodiments, the method includes venting the diversion and isolation chamber prior to drawing the initial volume of blood therein.

In some embodiments, the method further includes clamping a distal portion of the diversion and isolation chamber after the initial volume of blood is collected in the diversion and isolation chamber.

In some embodiments, the method includes coupling a first blood collection container to the blood collection interface after the initial volume of blood is collected in the diversion and isolation chamber, and collecting a first blood sample within the first blood collection container.

In some embodiments, the method includes removing the first blood collection container from the blood collection interface, coupling a second blood collection container to the blood collection interface, and collecting a second blood sample within the second blood collection container.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a plan view of a blood draw system with in-line vented blood diversion chamber in accordance with an aspect of the present disclosure;

FIG. 2 is a perspective view of the blood draw system of FIG. 1 coupled to a vascular access device and luer lock access device in accordance with an aspect of the present disclosure;

FIG. 3 a perspective view of the blood draw system of FIG. 1 coupled to a vascular access device, luer lock access device, and blood culture collection tube in accordance with an aspect of the present disclosure

FIG. 4 is a plan view of the in-line vented blood diversion chamber and luer lock access device of FIG. 2;

FIG. 5 is a plan view of the in-line vented blood diversion chamber, luer lock access device, and blood culture collection tube of FIG. 3; and

FIG. 6 is a plan view of a blood draw system with in-line vented blood diversion chamber in accordance with another aspect of the present disclosure.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw device in preparation for or during use. Similarly, in this application, the terms “in the distal direction” and “distally” mean in the direction toward an access connector portion of the fluid transfer device, and the terms “in the proximal direction” and “proximally” mean in the direction opposite the direction of the connector.

While not shown or described herein, it is to be understood that the blood sample collection systems described below may be utilized for blood draw from any suitable vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Embodiments of the present disclosure will primarily be described in the context of blood culture sample collection systems for use with PIVCs. However, embodiments of the present disclosure equally extend to use with other catheter devices.

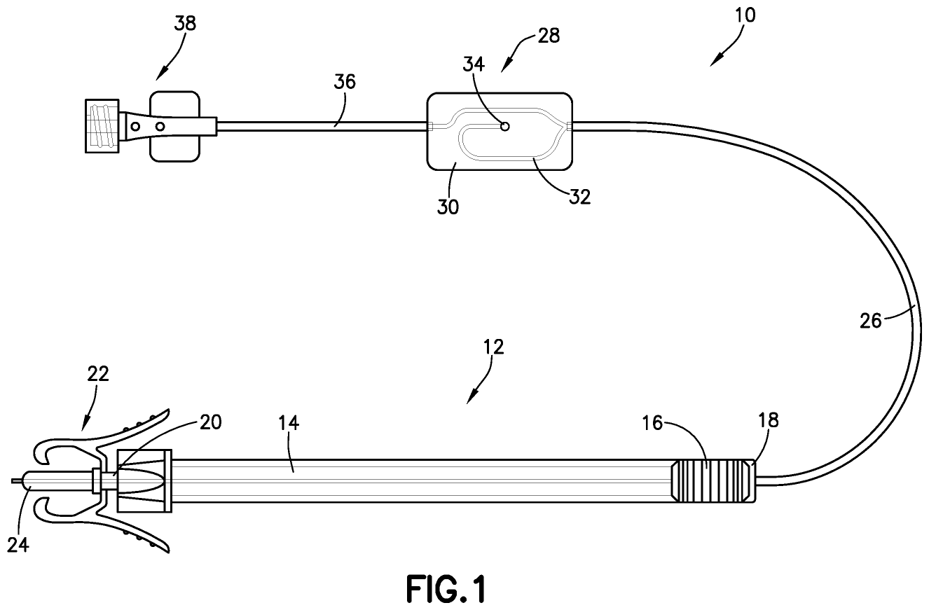

Referring to FIG. 1, a blood draw system 10 in accordance with an aspect of the present disclosure is shown. The blood draw system 10 includes a “line draw” blood draw device 12, which is configured to couple to a PIVC and includes a flexible probe (not shown) that is advanced through the PIVC, beyond the catheter tip, and into a vessel to collect a blood sample. After blood collection, the blood draw device 12 may be removed from the PIVC and discarded. One example of such a blood draw device is known as PIVO™ from Velano Vascular, Inc. However, it is to be understood that the blood draw system 10 is not limited to such a device, and any “line draw” blood draw device capable of drawing blood via an indwelling catheter may be utilized.

The blood draw device 12 includes an introducer body 14 and an actuator 16. The actuator 16 is configured to be linearly displaceable by a clinician along a track or other feature of the introducer body 14 between a proximal end portion 18 and a distal end portion 20 of the introducer body 14. The actuator 16 is operably coupled to an elongated, flexible probe or tube (not shown) to advance and retract the tube through a distal introducer portion 24 located near the distal end portion 20 of the introducer body 14. That is, displacement of the actuator 16 in a first (distal) direction advances the tube through the distal introducer portion 24 such that the tube can enter the patient's vasculature when blood draw device 12 is coupled to a vascular access device. Conversely, displacement of the actuator 16 in a second (proximal) direction retracts the tube through the distal introducer portion 24, ultimately retracting the tube from the patient's vasculature.

The blood draw device also includes a connector member 22 configured for selectively coupling the blood draw device 12 to, e.g., a needleless access connector of an adapter coupled to the vascular access device. In some embodiments, connector member 22 is configured as an alligator clip-type connector, with opposing distal clip portions allowing for securement of the blood draw device 12 to a surface of the needleless access connector. A pair of proximal clip portions are sized and configured to be pinched or otherwise manipulated by a clinician to release the distal clip portions from engagement with the needleless access connector.

Referring still to FIG. 1, a proximal extension tube 26 extends from the proximal end portion 18 of the introducer body 14, with the proximal extension tube 26 being fluidly coupled to the blood draw tube (not shown) of the blood draw device 12. Additionally, a proximal connector 38 such as, e.g., a luer port is coupled to the proximal end of the proximal extension tube 26, with the proximal connector 38 being configured for connection to, e.g., a luer lock access device or other blood collection interface.

In-line with the proximal extension tube 26 and between the blood draw device 12 and the proximal connector 38, blood draw system 10 includes an initial blood volume diversion device 28. As will be described in further detail below, the initial blood volume diversion device 28 is configured to divert and store an initial volume of blood that is drawn into the system via the blood draw device 12, as the initial volume of blood may be contaminated by, e.g., bacteria and/or fungi introduced during catheter insertion into the patient's skin and dermal layers.

In the embodiment shown in FIG. 1, the initial blood volume diversion device 28 includes a body 30, a vented diversion and isolation chamber 32, and a venting portion 34. In one embodiment, the venting portion 34 is configured to automatically vent air when the blood draw device 12 is coupled to a vascular access device and the blood draw tube of the blood draw device 12 is advanced into the patient's vasculature, thereby allowing an initial volume of blood to flow through the proximal extension tube 26 and to the vented diversion and isolation chamber 32. Alternatively, in other embodiments, the venting portion 34 may be manually vented when the blood draw device 12 is coupled to a vascular access device.

The vented diversion and isolation chamber 32 may have any shape or form with sufficient volume to divert and hold the initial blood volume. In some embodiments, the internal volume of the vented diversion and isolation chamber 32 is at least 0.15 mL. This volume is considered sufficient to capture the initial blood sample drawn into the system which may be contaminated. In some embodiments, the internal volume of the vented diversion and isolation chamber 32 is between 0.15 mL and 2.0 mL. In other embodiments, the internal volume of the vented diversion and isolation chamber 32 is between 0.15 mL and 5.0 mL. However, it is to be understood that the internal volume of the vented diversion and isolation chamber 32 is not limited to these examples.

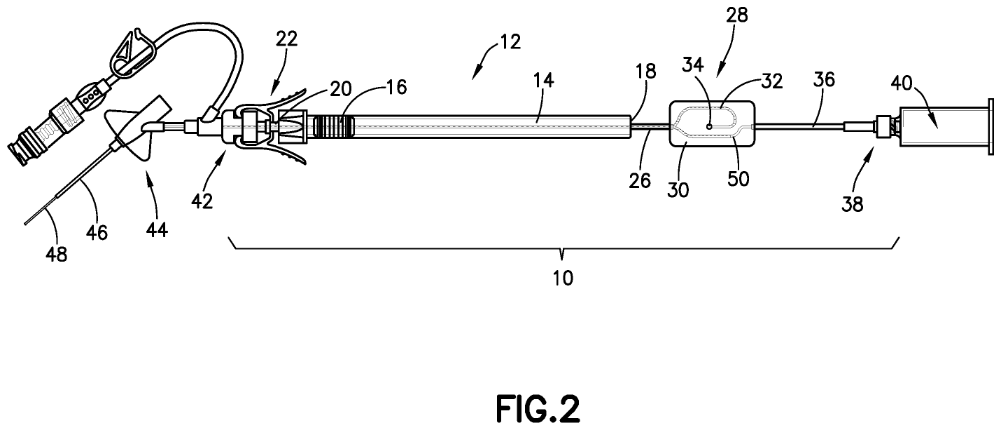

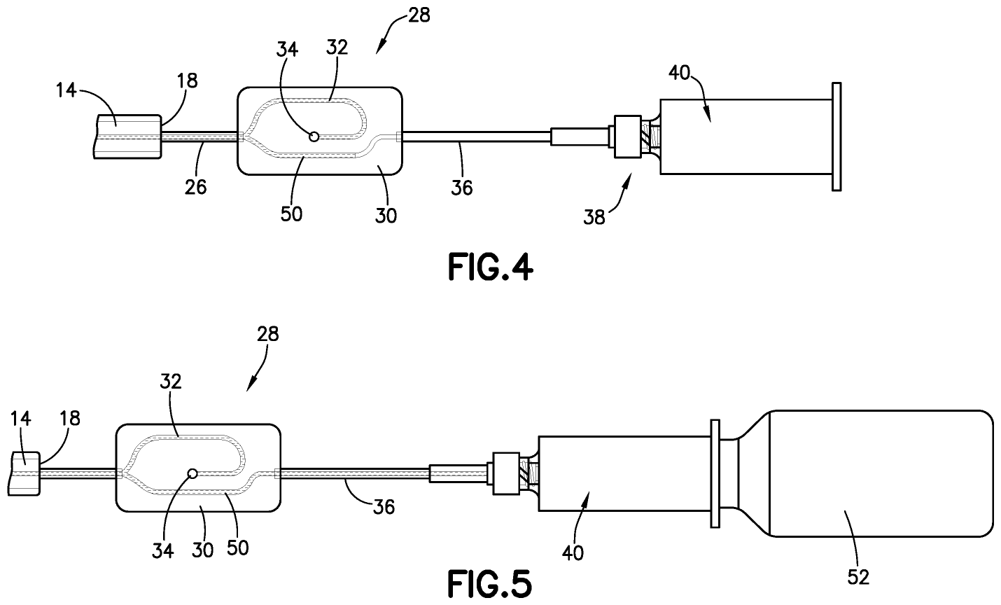

Referring now to FIGS. 2 and 4, the blood draw system 10 coupled to a vascular access device in a first configuration is illustrated. Specifically, the proximal connector 38 may be coupled to a luer lock access device 40, which is configured to receive any appropriate blood collection container and/or syringe for the collection of a blood sample therein. Additionally, the blood draw device 12 is coupled to, e.g., a near patient access port 42 via a needle-free connection. The near patient access port 42 is in fluid communication with a catheter adapter 44 having a catheter 46 extending distally therefrom. As noted above, the blood draw system 10 may be used with any appropriate vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

When the blood draw device 12 is initially coupled to the near patient access port 42, a blood draw tube 48 is not in an advanced position at or beyond the distal tip of the catheter 46, and no blood enters the blood draw device 12. However, the clinician may advance the blood draw tube 48 housed within the blood draw device 12 through both the catheter adapter 44 and the catheter 46 by distal advancement of the actuator 16 along the introducer body 14. At its fully-advanced position, the blood draw tube 48 extends to or beyond a distal tip of the indwelling catheter 46 and into a high blood flow location in the patient's vein, thereby providing a fluid path for venous blood to be drawn via the blood draw device 12.

With the blood draw tube 48 in this advanced position, the venting portion 34 of the initial blood volume diversion device 28 is vented, either automatically or manually. This venting of the venting portion 34, along with venous pressure, enables an initial volume of blood to flow through the tube 48, to the proximal extension tube 26, and into the vented diversion and isolation chamber 32 of the initial blood volume diversion device 28, with blood flow stopping at the venting portion 34, as is shown in FIGS. 2 and 4. When the vented diversion and isolation chamber 32 is filled with the initial volume of blood, a small portion of blood moves into a flashback visualization and blood sample fluid path arm 50 of the initial blood volume diversion device 28. In this way, the system 10 is primed with the initial volume of blood isolated within the vented diversion and isolation chamber 32, and the system is in condition for connection to, e.g., a blood culture vacuum tube for sample collection through the blood sample fluid path and the luer lock access device 40. However, because a collection container is not yet coupled to the luer lock access device 40, the flow of blood stops within the flashback visualization and blood sample fluid path arm 50 of the initial blood volume diversion device 28.

In some embodiments, air venting and blood flow into the initial blood volume diversion device 28 stops when blood contacts the venting portion 34. The venting portion 34 may be formed of, e.g., a membrane, paper, porous, film, or mechanical features that allows air to pass therethrough, but stops fluid from passing therethrough when wetted. When venting portion 34 is wetted, air is prevented from being pulled into the initial blood volume diversion device 28, which prevents the volume of blood held within the vented diversion and isolation chamber 32 from entering a blood sample fluid path portion 36 of the extension tube 26, thus preventing the potentially contaminated initial volume of blood from entering a blood collection container coupled to the luer lock access device. However, alternative means of isolating the initial blood sample within the initial blood volume diversion device 28 are also possible in accordance with other embodiments of the present disclosure. For example, in some embodiments, the initial blood sample may be manually isolated via a mechanical occlusion (i.e., via a clamp in a distal portion of the vented diversion and isolation chamber 32). In some embodiments, a one-way venting/fluid or check valve may be provided at the entrance of the vented diversion and isolation chamber 32, thereby allowing an initial blood sample to flow therein, but preventing outflow of the blood sample therefrom.

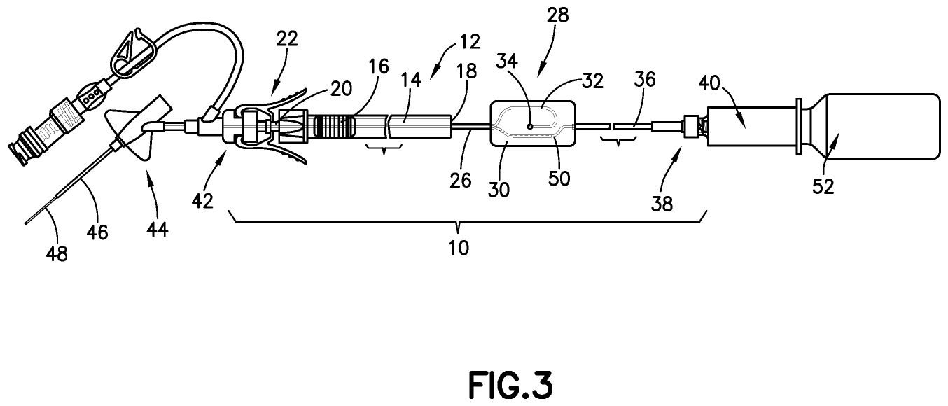

Next, referring to FIGS. 3 and 5, the blood draw system 10 coupled to a vascular access device in a second configuration is illustrated. In the second configuration shown in FIGS. 3 and 5, a blood collection container 52 is fluidly coupled to the luer lock access device 40, thereby drawing a blood sample from the patient's vasculature through the extension tube 26 via the flashback visualization and blood sample fluid path arm 50 of the initial blood volume diversion device 28. The blood collection container 52 may be any appropriate container such as, e.g., a BD BACTEC™ blood culture collection container, an evacuated tube, a syringe, etc. As noted above, the initial volume of blood is isolated within the vented diversion and isolation chamber 32, thereby preventing this potentially contaminated initial volume of blood from passing through the blood sample fluid path 36 and to the blood collection container 52, aiding in the prevention of false positive blood culture tests.

Once the desired blood sample is collected into the blood collection container 52, the blood collection container 52 may be disconnected from the luer lock access device 40 and sent for analysis. If additional blood samples are needed, one or more blood collection containers 52 may be coupled to the luer lock access device 40 to draw the desired samples. With each of these sample collections, the initial (and potentially contaminated) volume of blood remains within the vented diversion and isolation chamber 32 of the initial blood volume diversion device 28.

The blood draw system 10 described above with respect to FIGS. 1-5 is configured with a luer lock access device 40 and extension tube 26 to allow for greater flexibility during blood collection and to enable upright positioning of the blood collection container 52 during sample collection. In some embodiments, the blood draw system 10 can include a luer lock access device 40 having integrated extension tubing. In other embodiments, the luer lock access device 40 may have removably attached extension tubing. In some embodiments, the fluid path of all or some of the extension tube 26 may be optimized to reduce hemolysis during blood culture sample collection and/or during subsequent vacuum tube or syringe-based blood sample collections after the blood culture sample collection.

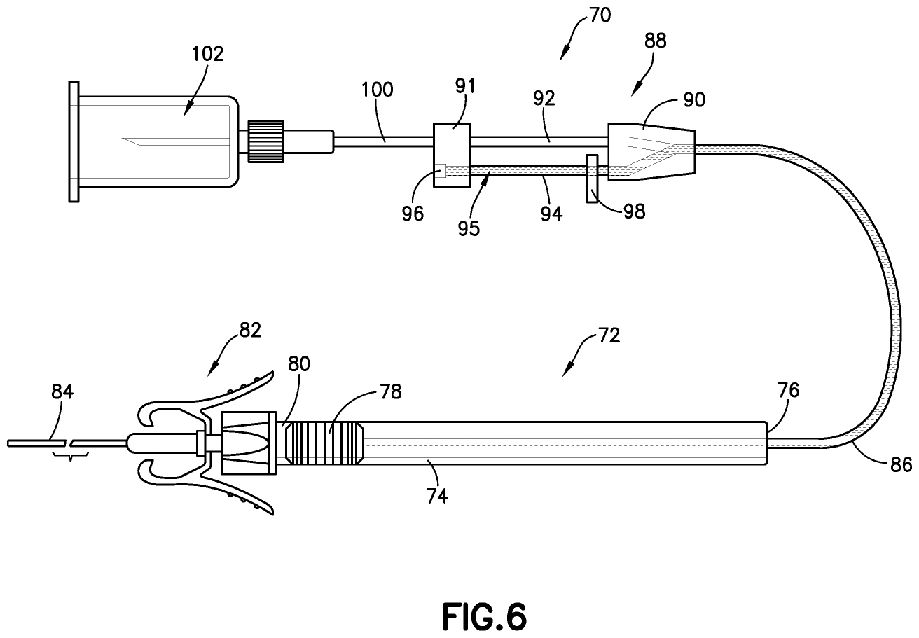

Next, referring to FIG. 6, a blood draw system 70 in accordance with another aspect of the present disclosure is shown. Similar to blood draw system 10 described above with respect to FIGS. 1-5, blood draw system 70 includes a “line draw” blood draw device 72, which is configured to couple to a PIVC (not shown) and includes a flexible probe or tube 84 that is advanced through the PIVC, beyond the catheter tip, and into a vessel to collect a blood sample. One example of such a blood draw device is known as PIVO™ from Velano Vascular, Inc. However, it is to be understood that the blood draw system 70 is not limited to such a device, and any “line draw” blood draw device capable of drawing blood via an indwelling catheter may be utilized.

The blood draw device 72 includes an introducer body 74 and an actuator 78. The actuator 78 is configured to be linearly displaceable by a clinician along a track or other feature of the introducer body 74 between a proximal end portion 76 and a distal end portion 80 of the introducer body 74. The actuator 78 is operably coupled to the blood draw tube 84 to advance and retract the tube 84 through a distal introducer portion located near the distal end portion 80 of the introducer body 74 when the actuator 78 is moved toward distal end portion 80, as is shown in FIG. 6. Conversely, displacement of the actuator 78 in a proximal direction retracts the tube 84 through the distal introducer portion, ultimately retracting the tube 84 from the patient's vasculature. The blood draw device also includes a connector member 82 configured for selectively coupling the blood draw device 72 to, e.g., a needleless access connector of an adapter coupled to the vascular access device. In some embodiments, connector member 84 is configured as an alligator clip-type connector, but is not limited as such, and may be any appropriate connector.

Referring still to FIG. 6, a proximal extension tube 86 extends from the proximal end portion 76 of the introducer body 74, with the proximal extension tube 86 being fluidly coupled to the blood draw tube 84 of the blood draw device 72. Additionally, a luer lock access device 102 is removably or non-removably coupled to the proximal end of the proximal extension tube 86. However, it is to be understood that other blood collection interfaces may be used, and blood draw system 70 is not limited to use only with a luer lock access device.

In-line with the proximal extension tube 86 and between the blood draw device 72 and the luer lock access device 102, blood draw system 70 includes an initial blood volume diversion device 88. The initial blood volume diversion device 88 is configured to divert and store an initial volume of blood that is drawn into the system via the blood draw device 72, as the initial volume of blood may be contaminated by, e.g., bacteria and/or fungi introduced during catheter insertion into the patient's skin and dermal layers.

In the embodiment shown in FIG. 6, the initial blood volume diversion device 88 includes a distal fluid path diversion adapter 90 and a proximal vented collar adapter 91. Between the distal fluid path diversion adapter 90 and a proximal vented collar adapter 91, a vented diversion and isolation chamber 94 is provided, with the vented diversion and isolation chamber 94 terminating at a venting portion 96 within the proximal vented collar adapter 91. Parallel to the vented diversion and isolation chamber 94, a primary flow tube 92 passes between the distal fluid path diversion adapter 90 and a proximal vented collar adapter 91, with the primary flow tube 92 being in fluid communication with the proximal extension tube 86 and the luer lock access device 102.

In one embodiment, the venting portion 96 is configured to automatically vent air when the blood draw device 72 is coupled to a vascular access device and the blood draw tube 84 of the blood draw device 72 is advanced into the patient's vasculature, thereby allowing an initial volume of blood to flow through the proximal extension tube 86 and to the vented diversion and isolation chamber 94. Alternatively, in other embodiments, the venting portion 96 may be manually vented when the blood draw device 72 is coupled to a vascular access device.

The vented diversion and isolation chamber 94 may have any shape or form with sufficient volume to divert and hold the initial blood volume. In some embodiments, the vented diversion and isolation chamber 94 is a rigid fluid chamber. In other embodiments, the vented diversion and isolation chamber 94 is a flexible chamber. In some embodiments, the internal volume of the vented diversion and isolation chamber 94 is at least 0.15 mL. This volume is considered sufficient to capture the initial blood sample drawn into the system which may be contaminated. In some embodiments, the internal volume of the vented diversion and isolation chamber 94 is between 0.15 mL and 2.0 mL. In other embodiments, the internal volume of the vented diversion and isolation chamber 94 is between 0.15 mL and 5.0 mL. However, it is to be understood that the internal volume of the vented diversion and isolation chamber 94 is not limited to these examples.

When the blood draw tube 84 of the blood draw device 72 is advanced through the catheter of a vascular access device (not shown), the venting portion 96 of the initial blood volume diversion device 94 is vented, either automatically or manually. This venting of the venting portion 96 enables an initial volume of blood 95 to flow through the tube 84, to the proximal extension tube 86, and into the vented diversion and isolation chamber 94 of the initial blood volume diversion device 88, with blood flow stopping at the venting portion 96, as is shown in FIG. 6. In this way, the system 70 is primed, with the initial volume of blood 95 isolated within the vented diversion and isolation chamber 94, and the system is in condition for connection to, e.g., a blood culture vacuum tube for sample collection through a blood sample fluid path 100 and the luer lock access device 102. However, because a collection container is not yet coupled to the luer lock access device 102, the flow of blood stops at the initial blood volume diversion device 88 in this initial configuration.

In some embodiments, air venting and blood flow into the initial blood volume diversion device 88 stops when blood contacts the venting portion 96. The venting portion 96 may be formed of, e.g., a membrane, paper, porous, film, or mechanical features that allows air to pass therethrough, but stops fluid from passing therethrough when wetted. When venting portion 96 is wetted, air is prevented from being pulled into the initial blood volume diversion device 88, which prevents the volume of blood held within the vented diversion and isolation chamber 94 from entering the primary flow tube 92 and the blood sample fluid path 100 of the extension tube 86, thus preventing the potentially contaminated initial volume of blood 95 from entering a blood collection container coupled to the luer lock access device 102. However, alternative means of isolating the initial blood sample within the initial blood volume diversion device 88 are also possible in accordance with other embodiments of the present disclosure. For example, as shown in FIG. 6, a fluid occlusion device 98 may be provided on a distal portion of the vented diversion and isolation chamber 94 to provide manual isolation of the initial volume of blood 95 via a mechanical occlusion. In other embodiments, a one-way venting/fluid or check valve may be provided at the entrance of the vented diversion and isolation chamber 94, thereby allowing an initial blood sample to flow therein, but preventing outflow of the blood sample therefrom.

While not shown in FIG. 6, in a second configuration of blood draw system 70, a blood collection container such as, e.g., a BD BACTEC™ blood culture collection container, an evacuated tube, a syringe, etc., is fluidly coupled to the luer lock access device 102, thereby drawing a blood sample from the patient's vasculature through the extension tube 86 via the primary flow tube 92 of the initial blood volume diversion device 88. As noted above, the initial volume of blood 95 remains isolated within the vented diversion and isolation chamber 94, thereby preventing this potentially contaminated initial volume of blood 95 from passing through the blood sample fluid path 100 and to the blood collection container, aiding in the prevention of false positive blood culture tests.

Using the blood draw systems 10, 70 described above with respect to FIGS. 1-6, numerous advantages over conventional methods of blood culture sample collection may be realized. First, the number of needle insertions for the patient may be reduced, thereby improving patient comfort and experience, as an existing vascular access device is used for blood culture sample collection. Additionally, using a blood draw device such as, e.g., PIVO™ from Velano Vascular, Inc. enables the blood draw tube to extend beyond the distal tip of the catheter, therefore reducing the risk of false positives from microbes that may be present in the catheter fluid path of an indwelling catheter.

Additionally, the blood draw systems have built-in automatic and passive diversion and capture of initial blood flow, which may be contaminated due to the presence of bacteria and/or fungi on the patient's skin, hair follicles, and dermal layers. This initial blood flow may be passively or manually isolated within a diversion chamber. Accordingly, the blood draw system eliminates the need for a separate blood discard sample to be collected, thereby reducing collection steps and improving workflow, while also reducing the opportunity for contamination during draw due to a reduced number of connections made with the blood draw system. Also, while existing blood culture collection devices with diversion chambers may only be used with a catheter immediately at the time of insertion, the blood draw systems described above may extend the use of blood collection devices with diversion chambers to any period throughout the catheter's dwell time.

The blood draw systems 10, 70 are also compatible with standard luer lock access devices, thereby enabling vacuum tube blood collection immediately after blood culture sample collection. Furthermore, the blood draw systems 10, 70 may be provided with optimized fluid paths for reduced hemolysis of the blood samples in subsequent blood collection samples after the initial blood culture sample collection.

While several embodiments of blood draw systems configured for blood sample collection from an indwelling catheter were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A blood draw system, comprising:
a blood draw device having a distal end portion and a proximal end portion, wherein the blood draw device comprises an actuator and a blood draw tube operably coupled to the actuator, wherein the actuator is configured to selectively advance the blood draw tube through a catheter of a vascular access device when the blood draw device is coupled to the vascular access device;
an extension tube extending from the proximal end portion of the blood draw device, wherein the proximal extension tube is in fluid communication with the blood draw tube of the blood draw device;
a proximal connector positioned at a proximal end of the extension tube; and
an initial blood volume diversion device positioned in-line with the extension tube between the blood draw device and the proximal connector, wherein the initial blood volume diversion device comprises a diversion and isolation chamber configured to receive and hold an initial volume of blood drawn through the extension tube by the blood draw device.

2. The system of claim 1, wherein the initial blood volume dispersion device further comprises a venting portion in fluid communication with the diversion and isolation chamber.

3. The system of claim 2, wherein the venting portion is configured to vent air when the blood draw device is coupled to the vascular access device and the blood draw tube of the blood draw device is advanced into a patient's vasculature to draw the initial volume of blood into the diversion and isolation chamber.

4. The system of claim 3, wherein the venting portion is configured to vent air automatically when the blood draw device is coupled to the vascular access device and the blood draw tube of the blood draw device is advanced into a patient's vasculature.

5. The system of claim 3, wherein the venting portion is manually vented when the blood draw device is coupled to the vascular access device and the blood draw tube of the blood draw device is advanced into a patient's vasculature.

6. The system of claim 3, wherein the venting portion is formed of at least one of a membrane, paper, porous material, film, or mechanical feature that allows air to pass therethrough but prevents fluid to pass therethrough when wetted.

7. The system of claim 1, wherein the initial blood volume diversion device further comprises a flashback visualization and blood sample fluid path arm, and wherein the flashback visualization and blood sample fluid path arm is in fluid communication with the extension tube and the proximal connector.

8. The system of claim 1, wherein the initial blood volume diversion device further comprises a distal fluid path diversion adapter and a proximal vented collar adapter, and wherein the diversion and isolation chamber extends between the distal fluid path diversion adapter and the proximal vented collar adapter.

9. The system of claim 8, wherein the proximal vented collar adapter includes a vented portion, and wherein a proximal end of the diversion and isolation chamber terminates at the vented portion.

10. The system of claim 8, wherein the initial blood volume diversion device further comprises a primary flow tube extending between the distal fluid path diversion adapter and the proximal vented collar adapter and positioned parallel to the diversion and isolation chamber, wherein the primary flow tube is in fluid communication with the extension tube and the proximal connector.

11. The system of claim 1, wherein the initial blood volume diversion device further comprises a fluid occlusion device positioned at a distal portion of the diversion and isolation chamber to selectively occlude the flow of blood from the diversion and isolation chamber.

12. The system of claim 1, wherein the proximal connector is configured to be removably coupled to a luer lock access device.

13. The system of claim 1, wherein the proximal connector is integrated with a luer lock access device.

14. The system of claim 1, wherein the blood draw device further includes an introducer body, and wherein the actuator is configured to move linearly along the introducer body to advance and retract the blood draw tube from the distal end portion of the blood draw device.

15. The system of claim 1, wherein the diversion and isolation chamber has an internal volume of at least 0.15 mL.

16. A method of using a blood sample collection system, comprising:
providing the blood sample collection system, the system comprising:
a blood draw device having a distal end portion and a proximal end portion, wherein the blood draw device comprises an actuator and a blood draw tube operably coupled to the actuator, wherein the actuator is configured to selectively advance the blood draw tube,
an extension tube extending from the proximal end portion of the blood draw device, wherein the proximal extension tube is in fluid communication with the blood draw tube of the blood draw device,
a blood collection interface positioned at a proximal end of the extension tube, and
an initial blood volume diversion device positioned in-line with the extension tube between the blood draw device and the blood collection interface, wherein the initial blood volume diversion device comprises a diversion and isolation chamber configured to receive and hold an initial volume of blood drawn through the extension tube by the blood draw device;
coupling the blood draw device to a vascular access device having an indwelling catheter;
advancing the blood draw tube of the blood draw device through the vascular access device and beyond a distal tip of the indwelling catheter; and
drawing the initial volume of blood through the blood draw tube and the extension tube and into the diversion and isolation chamber of the initial blood volume diversion device.

17. The method of claim 16, further comprising venting the diversion and isolation chamber prior to drawing the initial volume of blood therein.

18. The method of claim 16, further comprising clamping a distal portion of the diversion and isolation chamber after the initial volume of blood is collected in the diversion and isolation chamber.

19. The method of claim 16, further comprising:
coupling a first blood collection container to the blood collection interface after the initial volume of blood is collected in the diversion and isolation chamber, and
collecting a first blood sample within the first blood collection container.

20. The method of claim 19, further comprising:
removing the first blood collection container from the blood collection interface, coupling a second blood collection container to the blood collection interface, and
collecting a second blood sample within the second blood collection container.
